SEQUENCE LISTING (4 sequences)

SEQ ID NO: 1 - protein, 8 aa, Pangasius bocourti
NGTGPQNH

SEQ ID NO: 2 - protein, 10 aa, Pangasius bocourti
TAELEPLTDG

SEQ ID NO: 3 - protein, 12 aa, Pangasius bocourti
GRGGFGGRGGFG

SEQ ID NO: 4 - protein, 10 aa, Pangasius bocourti
LVMGFHPQYL

(19) United States
(12) Patent Application Publication
(10) Pub. No.: US 2022/0249622 A1
(43) Pub. Date: Aug. 11, 2022

(54) METHOD FOR PROTECTING BRAIN NEURON CELLS WITH BIOACTIVE COMPOUND

(71) Applicant: TCI CO., LTD., Taipei (TW)

(72) Inventors: YUNG-HSIANG LIN, Taipei (TW); YU-LING WANG, Taipei (TW)

(21) Appl. No.: 17/585,610

(22) Filed: Jan. 27, 2022

Related U.S. Application Data
(60) Provisional application No. 63/146,718, filed on Feb. 8, 2021.

(51) Int. Cl. A61K 38/39; A61P 25/28; A61K 35/60

(52) U.S. Cl. CPC A61K 38/39; A61K 35/60; A61P 25/28

(57) ABSTRACT

A method for protecting brain neuron cells of a subject in need thereof includes administering to the subject a composition including a bioactive compound. The bioactive compound is a peptide, and comprises at least one amino acid sequences as set forth in SEQ ID NO: 1 to SEQ ID NO: 4.

### CROSS-REFERENCE TO RELATED APPLICATION

This application claims the benefit of U.S. provisional application Ser. No. 63/146,718, filed on Feb. 8, 2021. The entirety of the above-mentioned patent application is hereby incorporated by reference herein and made a pan of the specification.

### REFERENCE OF AN ELECTRONIC SEQENCE LISTING

The contents of the electronic sequence listing (P211648USI_ST25 txt; Size: 913 bytes; and Date of Creation: Jan. 25, 2022) is herein incorporated by reference in its entirety.

### BACKGROUND

### Technical Field

The present invention relates to a method for protecting brain neuron cells, and more particularly to a method for protecting brain neuron cells with a peptide as a bioactive compound.

### Related Art

In recent years, the fish skin, which is known to be rich in collagen, has been widely used for manufacturing secondary processed food, gelatin, and the like.

Collagen is a very important protein in the human body, and widely exists in connective tissues. Collagen is the main component of human body ligaments, eye cornea, and other tissues, and is also a main component of an extracellular matrix. Collagen can enable the skin to maintain elasticity. Along with the loss of collagen, the wrinkles will appear on the skim

However, collagen products cannot be directly absorbed by the human body. In addition, the application of collagen is currently limited to die care of skin or knee joint which limits its coname.rcial value.

### SUMMARY

In view of this, in some embodiments, a use of a bioactive compound in preparing a composition for protecting brain neuron cells is provided. The bioactive compound is a peptide selected from the aroup consisting of amino acid sequences as set forth in SEQ ID NO: 1 to SEQ ID NO: 4.

In some embodiments, a method for protecting brain neurons is provided, including administering to a subject in need thereof a composition including a bioactive compound. The bioactive compound is a peptide, and the peptide comprises at least one amino acid sequence as set forth in SEQ ID NO: 1 tea SEQ ID NO: 4.

The bioactive compound or the composition including the bioactive compound has the effect of protecting brain neuron cells.

### BRIEF DESCRIPTION OF THE DRAWINGS

FIG. 1 is a graph showing the results (1) of relative activity of mitochondria after treatment on mouse cells by a peptide in sonic embodiments of the present invention;

FIG. 2 is a graph showing the results (2) of relative activity of mitochondria after treatment on mouse cells by a peptide in some embodiments of the present invention;

FIG. 3 is a graph showing the results of cell viability of human brain neuron cells which are damaged by β-amyloid after treated by a peptide in some embodiments of the present invention;

FIG. 4 is a graph showing the results of cell viability of human brain neuron cells which are damaged by MPP′ after treated by a peptide in some embodiments of the present invention;

FIG. 5 is a graph showing the results of correct answer rate of visual backward span test of an experimental group and a control group before and after drinking a composition containing a peptide in some embodiments of the present invention;

FIG. 6 is a graph showing the results of average reaction time of image memory test of an experimental group and a control group before and after drinking a composition containing a peptide in some embodiments of the present invention; and

FIG. 7 is a graph showing the results of correct answer rate of image memory test of an experimental group and a control group before and after drinking a composition containing a peptide in some embodiments of the present invention.

### DETAILED DESCRIPTION

The following will describe some specific implementations of the present. invention. Without departing from the spirit of the present invention, the present invention can still be practiced in many different forms, and the protection scope should not he limited to the conditions specified in this specification.

As used herein, the term “brain protection” refers to brain care, maintenance of brain cell health, or prevention of brain neuron damage and degeneration.

As used herein, the term “peptide” refers to a compound between amino acids and proteins, and is composed of many amino acids. In addition, a peptide as a bioactive compound may be an “isolated peptide” or a “synthetic peptide” The “isolated peptide” refers to a biologically active peptide fragment isolated from an organism or a derivative of an organism, The “synthetic peptide” refers to a biologically active peptide fragment synthesized according to a desired amino acid sequence by using an instrument or by manual experimental manipulation. In addition, the term “isolated peptide” mentioned herein is equivalent to “peptide isolated” or “peptide obtained through isolation”, and the term “synthetic peptide” is equivalent to “synthesized peptide” or “peptide obtained through synthesis”.

In some embodiments, a use of a. bioactive compound in preparing a composition for protecting brain neuron cells is provided. The bioactive compound is a peptide selected from the group consisting of ammo acid sequences as set forth in SEQ ID NO: 1 to SEQ ID NO: 4.

In some embodiments, a method for protecting brain neurons is provided, including administering to a subject in need thereof a composition including a bioactive compound. The bioactive compound is a peptide comprises at least one amino acid sequence as set forth in SEQ ID NO: 1 to SEQ ID NO: 4.

In some embodiments, the peptide is an isolated peptide prepared by carrying out an isolation step on a collagen peptide raw material. Preferably, in some embodiments, the collagen peptide raw material is a catfish skin collagen.

It should be understood that, as used herein, the term “collagen” is equivalent to “collagen protein”.

In some embodiments, the collagen peptide raw material may be a. commercially available catfish skin collagen peptide powder,

In some embodiments, the collagen peptide raw material may be a collagen extracted from catfish skin including proteins of fish skin cells (i.e., fish skin cell proteins) and proteins of fish flesh cells (i.e., fish flesh cell proteins) remaining on fish skin.

In some embodiments, a peptide as a bioactive compound is synthesized according to the amino acid sequences as set forth in SEQ NO: 1 to SEQ ID NO: 4 by using a peptide synthesizer or by artificial experiments.

In some embodiments, the peptide is a synthetic peptide prepared by concatenating the amino acid sequences as set forth in SEQ ID NO: I to SEQ ID NO: 4 by Fmoc solid-phase peptide synthesis.

In some embodiments, a peptide as a bioactive compound may be a group of peptides in which any of the amino acid sequences as set forth ira SEQ ID NO: 1 to SEQ ID NO: 4 are mixed together by chemical methods (such as enzymatic hydrolysis treatment) and/or physical methods (such as purification, isolation, hydrophilic and hydrophobic attraction, and polar and non-polar solvents).

In some embodiments, a peptide including at least one of the amino acid sequences as set forth in SEQ ID NO: 1 to SEQ ID NO: 4 can be used in preparing a composition for protecting brain neurons. The prepared composition can be used for reducing amyloid aqgregation to protect the brain neurons.

In some embodiments, a peptide including at least one of the ammo acid sequences as set forth in SEQ ID NO: 1 to SEQ ID NO: 4 can be used in preparing a composition for protecting brain neurons. The prepared composition can be used for inhibiting brain synapse damage to protect the brain neurons.

In some embodiments, a peptide including at least one of the amino acid sequences as set forth in SEQ 10 NO: 1 to SEQ ID NO: 4 can he used in preparing a composition for protecting brain neurons. The prepared composition can be used for enhancing activity of mitochondria to maintain the healthy state of cells and protect the brain neurons.

In some embodiments, a peptide including at least one of the ammo acid sequences as set forth in. SEQ ID NO: 1 to SEQ ID NO: 4 can he used in preparing a composition for protecting brain neurons. The prepared composition can have at least one of the following functions: improving image memory, improving cognitive ability, and increasing a correct answer rate.

In some embodiments, a. peptide including at least one of the amino acid. sequences as set forth in SEQ ID NO: 1 to SEQ ID NO: 4 can be used in preparing a composition for protecting brain neurons. The prepared, composition is a catfish skin collagen peptide powder.

The chemical reagents used in the following examples, unless otherwise noted, were purchased from Merck, Taiwan.

### EXAMPLE 1

### Peptide Isolation

First, 100 mg of catfish skin collagen peptide powder (purchased from Jellice, Singapore, lot number: CP-FC4, and its raw material was from basa fish (Pangasius bocourti)) was weighed and dissolved in 5 of buffer solution A, to obtain a collagen peptide solution. The buffer solution A contains 50 mM Tris/HCl buffer solution (pH 8.0) and 100 mM sodium chloride (NaCl) solution.

Then, the collagen peptide liquid was roughly isolated by using a fast performance liquid chromatograph (FPLC purification instrument, having a brand of ÄKTA GE :Healthcare Life Sciences, and called as a purification instrument hereinafter) to obtain a primary isolated peptide mixture. An isolation column disposed in the purification instrument was a molecular sieve colloid purification column (Superdex Peptide 10/300 GL, GE). The flow rate of the purification instrument was set to be 0.5 mUmin, and the wavelength of ultraviolet light for observation was set to be 280 nm or 220 nm. In addition, the primary isolated peptide mixture with a molecular mass less than 100 kDa and lypholizated at −80° C. (instrument brand: EYELA; model: FD-1000) for 12 h, so as to obtain a solid primary isolated peptide mixture.

30 mg of the solid primary isolated peptide mixture was taken and dissolved in 2 mL, of secondary deionized water containing 0.1% trill uoroacetic acid (LTA), so as to obtain a pre-isolated peptide mixture. Then, the pre-isolated peptide mixture was separated by a high performance liquid chromatogniphy (HPLC) system (machine type: Hitachi Chromaster HPLC system, brand: Hitachi, Tokyo, Japan) (called as an HPLC system hereafter), so as to obtain a plurality of groups of isolated peptides-An isolation column (model: TSKgel G2000SWXL brand: TOSOH BIOSCIENCE, 7.8×300 mm) was disposed in the HPLC system.

In set value of the HPLC system, 10 μL of pre-isolated peptide. mixture was separated with a separation buffer including a first solution (a solution with 0.05% TFA dissolved into water) and a second solution (a solution with 0.05% TFA dissolved into acetonitrile) according to a separation gradient. The separation gradient was set to be linearly ramped from the volume ratio of the first solution to the second solution being 0:100 to the volume ratio of the first solution to the second solution being 30:70 in 30 min: and the flow rate was set to be 0.5 milmin, the wavelength for observation was set to be 220 nm, and the column temperature. was set to be 30° C.

Finally, a plurality of groups of isolated peptides obtained by separation with HPLC system was lypholizated (instrument brand: EYELA; model: FD-1000) at −80° C. for 12 h, so as to obtain a plurality of groups of solid isolated peptides.

### EXAMPLE 2

### Sequence Identification of Isolated Peptides

The plurality of groups of solid isolated peptides obtained in Example 1 were subjected to protein identification. Firstly, after being prepared to reach a concentration of 20 mg/mL by deionized water. the plurality of groups of solid isolated peptides were subjected to protein identification by as liquid chromatography mass spectrometer (LC-MS/MS)

The LC-MS/MS was a quadrupole-time-of-flight (Q-TOF) tandem mass spectrometer system. The model of the liquid chromatography (LC) system was UltiMate 3000 RSLCnano LC Systems (brand: Thermo Fisher Scientific), and the model of the mass spectrometer was TripleTOF® 6600 System (brand: Applied Biosystems Sciex).

The separation column disposed in the liquid chromatography system was a C18 separation column (Acclaim PepMap C18, 75 μm I.D.×25 cm nanoViper, 2 μm, 100 Å column, brand: Thermo Fisher Scientific). solution system used by the LC-MS/MS includes a third solution (a solution with 0.1% formic acid dissolved into water) and a fourth solution (a solution with 0.1% formic acid dissolved into acetonitrile), The flow rate was set to be 300 nL/min. 10 μL of solid isolated peptides was dissolved in a buffer solution A, and then identification analysis was carried out under the separation gradient shown in Table 1.

TABLE 1

| Time | Content of third | Content of fourth |
|---|---|---|
| (min) | solution (%) | solution (%) |
| 0 | 95 | 5 |
| 4.5 | 95 | 5 |
| 31 | 65 | 35 |
| 32 | 10 | 90 |
| 52 | 10 | 90 |
| 53 | 95 | 5 |
| 70 | 95 | 5 |

In set values of the mass spectrometer, the survey scan was set to scan all ionized isolated peptides in the range of 100-15000 m/z (mass-to-charge ratio), in an information dependent acquisition (IDA) mode, the upper limit of detection of peptides was set to he 7500 daltons (Da). Then, these isolated peptides were analyzed to correspondingly generate multiple SIS /MS spectra, and these MS/MS spectra were searched in databases (NCB1 and UniProt) by a Mascot analysis program, so as to obtain amino acid sequences (shown in Table 2) and identification information (shown in Table 3) of these isolated peptides.

TABLE 2

| | | Molecular |
|---|---|---|
| Sequence number | Sequence | mass (Da) |
| SEQ ID NO: 1 | NGTGPQNH | 839.3522 |
| SEQ ID NO: 2 | TAELEPLTDG | 1044.4975 |
| SEQ ID NO: 3 | GRGGFGGRGGFG | 1080.5214 |
| SEQ ID NO: 4 | LVMGFHPQYL | 1203.6111 |

From Table 2, it could be known that in some embodiments, the molecular mass of the amino acid sequence of the isolated peptide was in a range of 800 Da to 1300 Da. In some embodiments, the amino acid sequence of the isolated peptide had 8 to 12 amino acids.

TABLE 3

| Sequence number | Identification information |
|---|---|
| SEQ ID NO: 1 | NAD kinase b |
| SEQ ID NO: 2 | NAD synthetase 1 |
| SEQ ID NO: 3 | Nucleolin |
| SEQ ID NO: 4 | Sestrin 1 |

From Table 3, it could be known that the amino acid sequence of the isolated peptide was a peptide fragment of the catfish skin. SEQ ID NO: 1 was a peptide fragment of NAD kinase b, SEQ ID NO: 2 was a peptide fragment of NAD synthetase 1. SEQ ID NO: 3 was a peptide fragment of nucleolin. SEQ ID NO: 4 was a peptide fragment of sestrin 1. Therefore, it could he known that the catfish skin collagen peptide powder in Example 1 is a composition includes peptides which having amino acid sequences as SEQ ID NO: 1 to SEQ ID NO: 4.

### EXAMPLE 3

### Peptide Synthesis

According to the amino acid sequences of 4 kinds of isolated peptides identified in Example 2, synthesized peptides were prepared by Fmoc solid-phase peptide synthesis using a peptide synthesis instrument (model: Focus XC III 0; brand: AAPPTEC).

The amino acid sequence of SEQ ID NO: 1 was taken as an example hereafter. According to Table 2, the amino acid sequence of SEQ ID NO: 1 was known as Asn-Gly-Thr-Gly-Pro-Gln-Asn-His.

Step (1): Firstly, resin was placed in a reaction tube, and according to a proportion of adding 15 mi., of dichloromethane (DCM) to per 1 g of resin, the resin was soaked in DCM for 30 min so that the resin expanded in the solution.

Step (2): The DCM was removed from the reaction tube. According to a proportion of adding 15 ml., of 20% piperidine dimethylformamide (piperidine DMF) solution to per 1 g of resin in the reaction tube, reaction was performed with the resin for 5 min, and then the solution was removed from the reaction tube. Then, according to a proportion of adding 15 mL of 20% piperidine dimethylformamide solution to per 1 g of resin in the reaction tube, reaction was performed with the resin again for 15 min, so as to remove the protecting qrotips on the resin, and obtain protecting-group-removed resin.

Step (3): After the solution was removed from the reaction tube again, a dozen grains of resin were taken out from the reaction tube for detection. Firstly, the resin was washed with ethanol three times, and then one drip of ninhydrin solution and one drip of phenol solution were added. The resin was heated at 105-110° C. for 5 min. When the ninhydrin solution and the phenol solution reacted with the resin and became dark blue, the result was positive reaction, indicating that the resin in the reaction tube was protecting-group-removed resin and can be combined with amino acids.

Step (4): According to a proportion of adding 10 mL of dimethylformamide to per 1 g of resin, add required volume of dimethylformamide (DMF) into the reaction tube after the step (3) to wash protecting-group-removed resin again,

Step (5): Excess protecting asparagine (Fmoc-Asn) and excess 1-hydroxybenzotriazole (HOBt) were dissolved by a small amount of DMF, and the solution was added into the reaction tube containing the protecting-group-removed resin for reaction for 90 min. The excess mean that the volume used is more than three times the volume of DMF.

Step (6): According to a proportion of adding 10 mL of dimethylformamide to per 1 g of resin, add required volume of dimethylformamide (DMF) into the reaction tube after the step (5) to wash the resin connected with the amino acids in the reaction tube.

Then, Step (2) to Step (6) above were repeated until the other amino acids (Gly, Thr, Gly, Pro, Gin, Asn, and His) were sequentially connected to form a primary synthesized peptide with an amino acid sequence of SEQ ID NO; 1.

Step (7): According to a proportion of adding 10 mL of dimethylformamide to per 1 g of resin in the reaction tube, add required volume of dimethylformamide (DMF) into the reaction tube after the Step (6) to wash the primary synthesized peptide in the reaction tube. Then, dichloromethane and ethanol were added to the reaction tube in the same volume as DMF to wash the primary synthesized peptide again,

Step (8): The washed primary synthesized peptide from step (7) was reacted with 10 mL of lysis solution for 120 min to separate the primary synthesized peptide from the resin. The lysis solution contains 86% of TFA, 4% of thioanisole, 3% of water, 5% of ethane-1,2-dithiol (EDT), and 2% of phenol.

Step (9): The residual resin in lysis solution was removed by a sand core funnel, so as to obtain a secondary synthesized peptide. Then, the secondary synthesized peptide was subjected to suction filtration using a Büchner funnel. During the suction filtration, 80 mL, of diethyl ether was added into the Buchner funnel to wash the secondary synthesized peptide, so as to obtain a third synthesized peptide. The third synthesized peptide was a solid.

Step (10): After 1 mL of the third synthesized peptide was dissolved by 0.5 mL of deionized water, and then separated and purified by an HPLC system (model: Hitachi Chromaster HPLC system; brand: Hitachi, Tokyo, Japan), so as to obtain a pure synthesized peptide.

In the HPLC system. a C18 column (brand: Gemini-NX) was used and the detection wavelength was set to be 220 nm. In addition, the elution was carried out with an chant, which contains a fifth solution (a solution with 0.1% TEA dissolved into water) and a sixth solution (a solution with 0.1% TFA dissolved into aceionitrile) mixed in a separation gradient. The separation gradient was set to the volume ratio of the fifth solution to the sixth solution of 100:0 and was linearly increased to the volume ratio of 30:70 within 30 min. The flow rate was set to be 1 mL/min. In addition, a result that the purities of the synthesized peptide reached 95% or above could be obtained by calculating the peak area of the synthesized peptide according to the HPLC. chromatography. Therefore the synthesized peptide including the amino acid sequence SEQ ID NO: 1 could be obtained.

Likewise, other amino acid sequences (SEQ ID NO: 2 to SEQ ID NO: 4 could also be treated according to the above process. After Step (1), the above Step (2) to Step (4) were repeated. According to the amino acid sequences shown in Table 2, the required amino acid raw materials were correspondingly added to synthesize the peptides of SEQ ID NO: 2 to SEQ ID NO: 4 in Step (5) and Step (6). Then, Step (7) to Step (10) were performed for washing and purification, so as to obtain purify (the purity is as hid as 95%) synthesized peptide (i.e., SEQ ID NO: 2 to SEQ ID NO: 4).

### EXAMPLE 4

### Experiment on Activity of Mitochondria of Neuron Cells Affected by Bioactive Compound

Materials and instruments:

1. Cell strain: mouse brain neuroblastoma. cells Nero2a, from the American Type

Culture Collection (ATCC®, Cat. CCL-131), hereinafter referred to as Nero2a cells.

2. Culture medium: Dulbecco's modified Eagle's medium (DMEM, purchased from Gibco, Cat. 11965-092) containing 10 vol % of Fetal Bovine Serum (FBS, purchased from Gibco, 10437-028) and 3.7 g/L of sodium bicarbonate (purchased from Sigma, S5761-500G).

3. Phosphate buffered saline (hereinafter referred to as PBS solution): purchased from Gibco, product No. 10437-028.

4. Mitochondrial n embranepotential assay kit (MitoScreen (.IC-1) kit purchased from BD, model 551302): with ,IC-1 dye (lyophilized) and 10× assay buffer. Before use, the assay buffer was diluted 10-fold with 1 PBS solution to form 1× assay buffer, 130 μl of dimethyl sulfoxide (DMSO) was added into the JC-1 dye (lyophilized) to form a JC-1 stock solution. Then, the stock solution was diluted to 1/100 with the 1 × assay buffer to form a JC-1 working reagent

5. Trypsin: Trypsin-EDTA (purchased from Gibe( ) diluted l -fold with 1× PBS solution.

6. Flow cytometer: purchased from BD Pharmingen, model BDTM Accuri C6 Plus.

Experimental steps:

The Nero2a cells were seeded into a 6-well culture plate at a density of 1.5×10 cells per well, and 2 ml., of the above-mentioned fresh culture medium was added to each well and incubated for 24 hours at 37° C. and 5% CO₂.

Remove the culture medium from each well. The Nero2a cells were divided into a control group and experimental groups 1-5. Control group was added with the fresh culture medium. Experimental group I was added with fresh culture medium containing 50 μg/mL synthesized peptide of SEQ .117 NO :1; experimental group 2 was added with fresh culture medium containing 50 μg/mL synthesized peptide of SEQ ID NO:2; experimental group 3 was added with fresh culture medium containing 50 μg/mL synthesized peptide of SEQ ID NO 3, experimental group 4 was added with fresh culture medium containing 25 μg/mL synthesized peptide of SEQ ID NO:4; and experimental group 5 was added with fresh culture medium containing 1 mg/mL of catfish skin collagen peptide powder from Example. 1. Each group was triplicated and incubated at 37C. for 24 hours.

In each group, the culture medium was removed, and then the cells were washed twice with the PBS solution. Then, 200 μL of trypsin solution was added to react with the cells for 3 min, and 6 mL of fresh culture medium was added to stop the reaction.

The suspended cells and culture medium were collected into a 1.5 mL centrifuge tube and centrifuged at 400 g for 10 min to precipitate the cells. Then, the precipitated cells collected by centrifugation were resuspended in PBS solution to form a cell suspension, and the cell suspension was transferred into a 1.5 mL centrifuge. tube. Then, the cell suspension was centrifuged again at 400 g for 5 min, and then a supernatant thereof was removed. Then, 100 μL of JC-1 working reagent was added into each centrifuge tube.

The cell precipitate in each centrifuge tube was vortexed with the JC-1 working reagent uniformly and incubated for 15 mM under light-proof treatment,

After incubation in darkness, each centrifuge tube was centrifuged at 400 g for 5 min and the cells were washed twice with PBS solution. Finally, 500 of 1X PBS was added to each centrifuge tube to re suspend the cells, so as to obtain the cell solution to be tested.

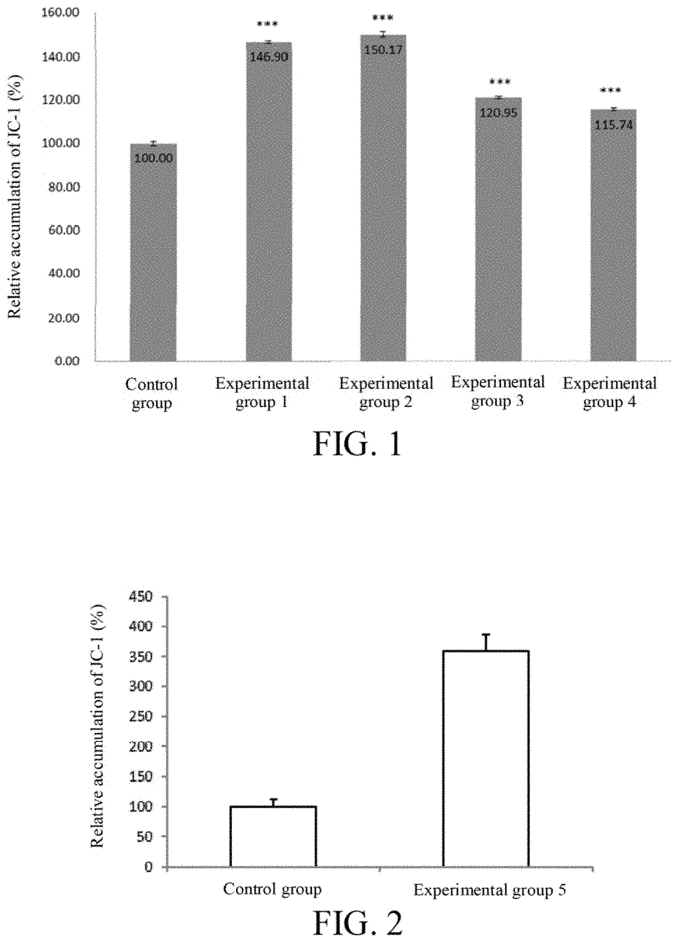

The cell solution of the control group and the cell solution of each experimental group were analyzed for the activity of mitochondria by using a flow cytometer. The experimental results are shown in FIG. 1 and FIG. 2,

Standard deviations were calculated by an STDEV formula of Excel software, and whether statistically significant differences existed or not was analyzed by one-tail student t-test in the Excel software. The values for each group in FIGS. 1 and 2 are the mean ±standard deviation taken from the triplicate experiments. The p value of each group was calculated compared to the control group. In the figures, “*” represents a p value less than 0.05, “**” represents a p value less than 0.01, and “***” represents a p value less than 0.001, More “*” represents more significant statistical differences.

Taking the value of the control group as 100% of relative accumulation of JC-1, the value of each experimental group was converted into relative accumulation of JC-1 (%).

Referring to FIG. 1 and FIG. 2, the relative accumulation of JC-1 of the experimental group 1 was 146.90%; the relative accumulation of JC-1 of the experimental group 2 was 150,17%; the relative accumulation of IC-1 of the experimental group 3 was 120.95%; the relative accumulation of JC-1 of the experimental group 4 was 115.74%; and the relative accumulation of JC-1 of the experimental group 5 was 362%. According to the results of this experiment, the activity of mitochondria of neuron cells was significantly increased in experimental groups 1 to 5 compared to the control group, especially in the experimental group 5, the cells treated with the catfish skin, collagen peptide powder containing the peptide of SEQ ID NO: 1 to SEQ ID NO: 4 showed a significant increase in the activity of mitochondria by 3.6-fold. This also indicated that the peptides with the amino acid sequences cif SEQ ID NO: 1 to SEQ ID NO: 4 and their combinations increased the activity of mitochondria of neuron cells.

A composition prepared with a peptide having one or more ammo acid sequences as set forth in SEQ ID NO: 1 to SEQ ID NO: 4 also can increase the activity of mitochondria. The increased activity of mitochondria helps cells to maintain the healthy state. Therefore, a composition containing a peptide having one or more amino acid sequences as set forth in SEQ ID NO: 1 to SEQ ID NO: 4 has an effect of protecting brain neuron cells to achieve brain protection.

### EXAMPLE 5

### Experiment of Inhibitory Effect of Bioactive Compound on Brain Neuron Damage Caused by 15-Amyloid

Materials and instruments:

1. Cell strain: human brain neurons SH-SYSY from the American Type Culture Collection (ATCC®, Cat. CRL-2266), hereinafter referred to as SH-SYSY cells.

2. Culture medium: Dulbecco's modified Eagle's medium (DMEM, purchased from Gibco, Cat, 11965-092) containing 10 vol % of Fetal Bovine Serum (FBS, purchased from Gibco, 10437-028) and 3.7 of sodium bicarbonate (purchased from Sigma, S 5761 -500G).

3. Phosphate buffered saline (hereinafter referred to as PBS solution): purchased from Gibco, product No 10437-028.

4. β-amyloid: purchased from Sigma-Aldrich, product No. A9810. A β-amyloid stock reagent with a concentration of 0.25 mg/mL was prepared from a powder. During the experiment, the concentration of β-amyloid stock reagent was diluted to 5 uM with fresh culture medium for use.

5. Methyllycaconitine (MLA): purchased from Sigma-Aldrich, product No. M168. An MLA stock reagent with a concentration of 5 mM was prepared from a powder. During, the experiment, the concentration of MLA stock reagent was diluted to 5 μM with fresh culture medium for use.

6. ELISA reader: brand. BioTek, model: FLx800,

Experimental steps:

The SH-SYSY cells were seeded at a density of 5×10³ cells per well into a 96-well culture plate and incubated for 24 hours at 37° C. and 5% CO₂.

The culture medium was removed from each well. The SH-SYSY cells were divided into a blank group, a negative control group, a positive control group, and an experimental group. The fresh culture medium was added into the wells of the blank group. The fresh culture medium containing 5 μM β-amyloid was added into the wells of the negative control group. The fresh culture medium containing 5 μM β-amyloid and 5 μM MLA was added into the wells of the positive control group. The fresh culture medium containing 5 μM β-amyloid and 1 mg/mL catfish skin collagen peptide powder from Example 1 was added into the wells of the experimental group. Each group was triplicated and incubated at 37° C. for 72 hours.

Then, 15 μL of 5 mg/mL MTT apoptosis detection solution (3-(4,5-dimethylthiazol-2-yl)-2,5-diphenyltetrazolium bromide) was added to each group for 3-4 h at 37° C. and 5% CO₂.

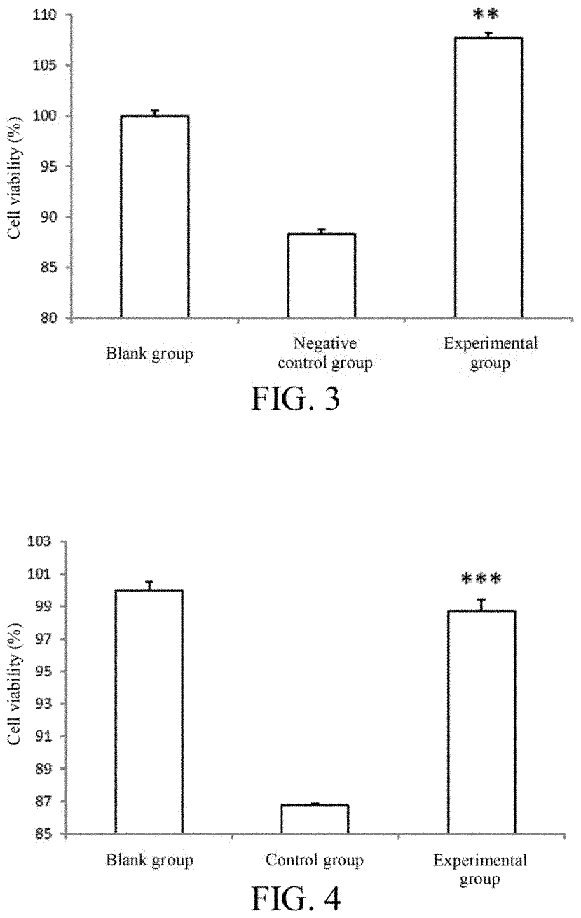

The culture medium in each well was removed. in each group, 50 μL of Dimethyl sulfoxide (DMSO) was added to dissolve the purple crystals of formazan produced by the redox reaction to produce a. purple liquid, Finally, an absorbance (at 570 nm) of each group was measured by an ELISA reader to analyze the cell viability of each group. The experimental results are shown m FIG. 3.

Standard deviations were calculated by an STDEV formula of Excel software, and whether statistically significant differences existed or not was analyzed by one-tail student t-test in the Excel software. The values for each group in FIGS. 3 are the mean standard deviation taken from the triplicate experiments. The p value of each group was calculated compared to the control group. In the figures, “*” represents a p value less than 0.05, “**” represents ai p value less than 0.01, and “***” represents a p value less than 0.001. More “*” represents more significant statistical differences.

Taking the value of the blank group as 1.00% of cell viability, each value of the negative control group, the positive control group, and the experimental group was converted into cell viability (%).

Referring to FIG. 3, the cell viability of the negative control group was 88%, and the cell viability of the experimental group was 108%. According to the results of the negative control group, when brain neurons were subjected to β-amyloid, cell damage was caused and cell viability was reduced (by about 12%). However, according to the results of the experimental group, the presence of peptides of SEQ ID NO:1 to SEQ ID NO.4 provided protection to the neuron cells when they were damaged by β-amyloid, which increased the cell viability to 108%. In addition, the cell viability of the experimental group was 20% higher than that of the negative control group.

Since β-amyloid is the main component of the amyloid plaques found in the brains of patients with Alzheimer's disease, the p-arnyloid can be used as a drug model of Alzheimer's disease.

Therefore, it is clear that catfish collagen peptides have a protective function on brain neuron cells. In other words, a composition containing the peptide of SEQ IID NO: 1 to SEQ ID NO: 4 can inhibit the damage to brain neurons caused by β-amyloid, and can be considered as a potential drug or a food for special health use for preventing brain diseases, especially preventing the occurrence of Alzheimer's disease.

### EXAMPLE 6

### Experiment of Inhibitory Effect of Bioactive Compound on Brain Neuron Damage Caused by 1-methyl-4-phenylpyridin-1-ium (MPP+)

The cells, culture medium, MTT apoptosis detection solution, ELISA reader, and statistical method of cell viability used in this example are the same as in Example 5, and details are not described herein again.

Experimental steps:

The SII-SYSY cells were seeded at a density of 5×10³ cells per well into a 96-well culture plate and incubated for 24 hours at 37° C. and 5% CO₂.

The culture medium was removed from each well. The SH-SYSY cells were divided into a blank group, a control group, and an experimental group. The fresh culture medium was added into the wells of the blank group. The fresh culture medium containing 5 μM MPP+ was added into the wells of the control group. The fresh culture medium containing 5 μM MPP+ and 1 mg/mL catfish skin collagen peptide powder from Example 1 was added into the wells of the experimental group. Each group was triplicated and incubated at 37° C. for 48 hours.

Then, 15 μL of 5 MTT apoptosis detection solution was added to each group for 3-4 h at 37° C. and 5% CO₂.

The culture medium in each well was removed. In each group, 50 μL of DMSO was added to dissolve the purple crystals of formazan. Finally, an absorbance (at 570 nm) of each group was measured by an ELISA reader to analyze the cell viability of each group. The experimental results are shown in FIG. 4.

Taking the value of the blank group as 100% of cell viability, each value of the control group and the experimental group was converted into cell viability (%).

Referring to FIG. 4, the cell viability of the control group was 86%, and the cell viability of the experimental group was 99.8%. According to the results of the control group, when brain neurons were subjected to MPP+, cell damage was caused. The cell viability in the control group was reduced by 14%. However, according to the results of the experimental group, the presence of peptides of SEQ ID NO: 1 to SEQ ID NO:4 provided protection to the brain nerve cells when the brain nerve cells were damaged by MPP+, and the cell viability was increased to 99.8%. in addition, the cell viability of the experimental group was 13.8% higher than that of the control group, with a statistically significant difference.

MPP+ is toxic because it affects oxidative phosphorylation in mitochondria, causing ATP depletion and cell death. Moreover, according to the literature, MPP+ can be absorbed into brain neurons through the dopamine transporter on the synaptic cell membrane, thereby causing synaptic damage. Therefore, MPP+ can be used as a drug model of Parkinson's disease.

However, according to the experimental results of Example 6, the experimental group can enhance the cell viability and has a function of protecting brain neurons. In other words, a composition containing the peptide of SEQ ID NO: 1 to SEQ ID NO: 4 can inhibit the damage to synapses caused by MPP+, and therefore can be considered as a potential drug or a food for special health use for preventing brain diseases, especially preventing the occurrence of Parkinson's disease.

### EXAMPLE 7

### Human Efficacy Experiment of Composition Containing B.toactive Compound

Test sample: Each 3 g packet contains more than 90% of catfish skin collagen peptide powder from Example 1.

Control sample: Each 3g packet contains more than 90% of tilapia collagen, purchased from Rousselot (product number: F2000 HD), and its raw material was from tilapia.

Number of subjects: 16 subjects aged 25-35 years that were diagnosed by major medical institutions as having nervousness, anxety, or memory loss. 8 subjects were randomly assigned to the experimental group and consumed one packet of the test sample per daily; and the remaining 8 subjects were assigned to the control group and consumed one packet of the control sample per daily.

Experimental method: Each subject was prohibited from using any caffeine-based stimulant beverage 24 hours prior to the test. During the test period, each subject consumed a daily sample of the designated drink for their group for 4 weeks. Visual backward span test was carried out on each subject before consumption (at week 0) and after 4 weeks of consumption (at week 4).

Detection items include: visual backward span est and image memory test.

(1) Results of visual backward span test

Through the visual backward span test on the website http://cognitivefun.net/test/10, a number with two or more digits, such as the number “514”, was randomly displayed on a display screen in front of each subject within a certain period of time. Then, the subject was asked to reversely input the number he/she just saw, such as “415”. The response time of each subject to input the number and the correct answer rate of each subject were recorded.

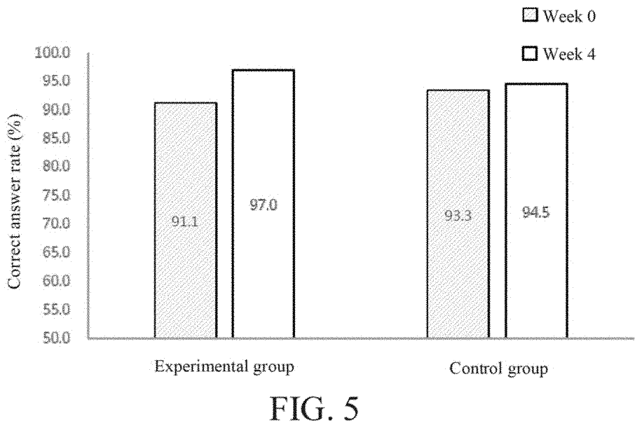

Each subject was subjected to the visual backward span test before consumption (at week 0) and after 4 weeks of consumption (at week 4), and then the correct answer rate (%) of each group was calculated, The results are shown in FIG. 5. At week 0, the correct answer rate of the experimental group was 91.1%, and the correct answer rate of the control group was 93.3%. At week 4, the correct answer rate of the experimental group was 97.0%, and the correct answer rate of the control group was 94.5%. Therefore, drinking the composition containing peptides of SEQ ID NO:1 to SEQ ID NO:4 has an effect on memory enhancement.

(2) Results of image memory test

Through the image memory test on the website https://memtrax.com/test/, images were displayed continuously on a display screen w front of each subject within a certain period of time. During the display, when a subject saw a currently displayed image that was the same as the previous one, the subject pressed the space bar or clicked on the image displayed on the screen to complete the answering. The response time of each subject to complete the answering and the correct answer rate of each subject were recorded.

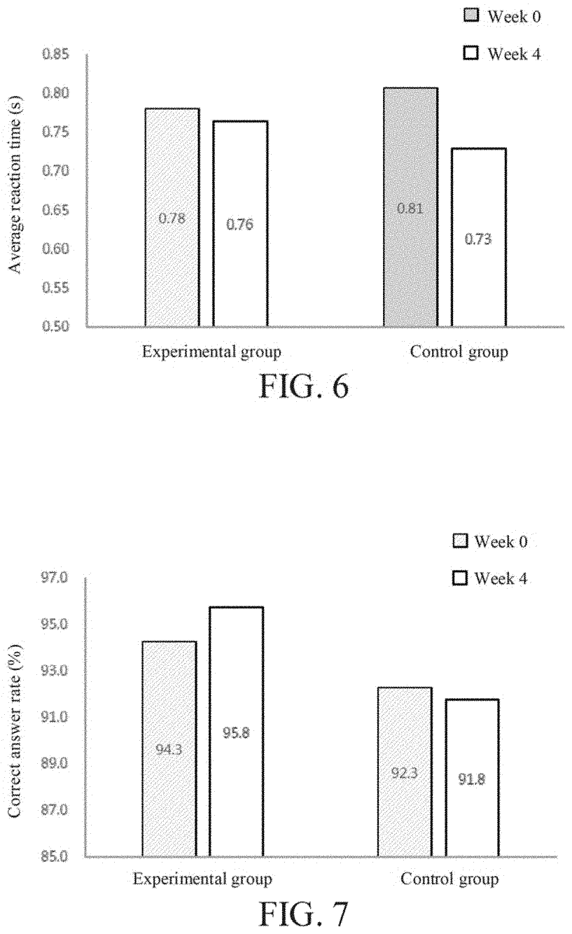

Each subject was subjected to the image memory test before consumption (at week 0) and after 4 weeks of consumption (at week 4), and then the average response time of each group was calculated. The results are shown in FIG. 6. At week 0, the average reaction time of the experimental group was 0,78 s, and the average reaction time of the control group was 0.81 s. At week 4. the average reaction time. of the experimental aroup was 0.76 s, and the average reaction time of the control group was 0.

Each subject was subjected to the image memory test before consumption (at week 0) and after 4 weeks of consumption (at week 4), and then the correct answer rate (%) of each group was calculated. The results are shown in FIG. 7. At week 0, the correct answer rate of the experimental group was 94.3%, and the correct answer rate of the control group was 92.3%. At week 4, the correct answer rate of the experimental group was 95.8%, and the correct answer rate of the control group was 91.8% Therefore, drinking the composition containing peptides of SEQ ID NO:1 to SEQ ID NO:4 has an effect on image memory enhancement.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method for protecting brain neuron cells in a subject in need thereof, comprising administering to the subject a composition comprising a bioactive compound, wherein the bioactive compound is a peptide, the peptide comprises at least one amino acid sequence as set forth in SEQ ID NO: 1 to SEQ ID NO: 4.

2. The method according to claim 1, wherein the composition is used for reducing amyloid aggregation to protect the brain neuron cells.

3. The method according to claim 1, wherein the composition is used for is brain synapse damage to protect the brain neuron cells.

4. The method according to claim 1, wherein the composition is used for enhancing activity of mitochondria to protect the brain neuron cells.

5. The method according to claim 1, wherein the composition has at least one of the following functions: improving image memory and improving cognitive ability.

6. The method according to claim 1, wherein the composition is a catfish skin collagen peptide powder.

7. The method according to claim 1, wherein the peptide is an isolated peptide prepared by carrying out an isolation step on as collagen peptide raw material.

8. The method according to claim 7, wherein the collagen peptide raw material is a catfish skin collagen.

9. The method according to claim 7, wherein the isolation step comprises:
a purification step: purifying the collagen peptide raw material by using fiist performance liquid chromatogntphy to obtain a purified product; and
a separation step: separating the purified product by high performance liquid chromatography under an elution condition comprising: eluting the purified product with acetonitrile containing 0.05% TFA and water containing 0.05% TFA in an elution gradient to obtain the isolated peptide.

10. The method according to claim 1, wherein the peptide is a synthetic peptide prepared by concatenating the amino acid sequences as set forth in SEQ ID NO: 1 to SEQ ID NO: 4 by Fmoc solid-phase peptide synthesis.
